(19) United States
(12) Patent Application Publication
(10) Pub. No.: US 2011/0318819 A1
(43) Pub. Date: Dec. 29, 2011

(54) DEVICE FOR CULTIVATING ALGAE AND/OR MICROORGANISMS FOR TREATING AN EFFLUENT, AND BIOLOGICAL FRONTAGE

(75) Inventors: Anouk Legendre, Paris (FR); Nicolas Desmazieres, Paris (FR)

(73) Assignee: X'TU, Paris (FR)

(21) Appl. No.: 13/141,425

(22) PCT Filed: Dec. 22, 2009
(86) PCT No.: PCT/FR2009/001474

§ 371 Date: Sep. 13, 2011

(30) Foreign Application Priority Data
Dec. 23, 2008 (FR) 08291237.9

(51) Int. Cl. C12M 1/38; C12M 1/24; C12M 1/04; C12M 1/00

(52) U.S. Cl. 435/286.1; 435/289.1; 435/292.1; 435/304.1

(57) ABSTRACT

Device for processing effluents includes: a container for cultivating algae and/or microorganisms in an aqueous medium; supply elements for the algae and/or microorganism culture; elements for injecting an effluent into the algae and/or microorganisms, the effluent coming from a building; elements for adjusting the temperature of the algae and/or microorganism culture; effluent recovery elements for recovering the effluent from a building and for injecting the same into the algae and/or microorganism culture; and optionally a lighting system for promoting the growth of the algae and/or microorganism culture. The device can be used for producing biofuel, organic molecules, chemical compounds and proteins. The biofuel thus obtained may consist of oleaginous biomass, for example, which can directly be used in a thermal power plant or be converted by pyrolysis into coal or biopetroleum. The device makes it possible to implement a new biological frontage and biological reactors integrated in the frontages of buildings under construction or already existing.

### TECHNICAL FIELD

The present invention relates to a device for treating effluents and/or for example, making a building frontage capable of producing primary biochemical energy obtained by photosynthesis. The device of The present invention can be used for producing biofuel, organic molecules, chemical compounds and proteins.

The biofuel thus obtained may consist of oleaginous biomass, for example, which can be directly used in a thermal power plant or be converted by pyrolysis into coal or biopetroleum.

The invention can be used directly for the energy needs of the building and supply its own power plant and/or be exported and/or sold.

The present invention also makes it possible to implement a new biological frontage and biological reactors integrated in the frontages of modern or old buildings, under construction or already existing.

### STATE OF THE ART

Cities pollute and produce lots of carbon dioxide from their thermal plants, boiler rooms of buildings, cold production plants. Moreover, the treatment of foul air from buildings is not always easy, especially when it emanates from humans, as well as from underground parks, especially car parks.

Carbon dioxide (CO₂) and Nitrogen Oxide (NO₂) in particular escape into the atmosphere and contribute to the greenhouse effect and climate degradation.

Cities also consume a lot of energy which is produced remotely, and must be transported at a high cost with a significant pressure drop even for electrical energy. Fully air-conditioned business areas using electrical energy via thermal plants drain a lot of energy.

Many solutions have been examined on how to treat these gases today called effluents; especially filters, air purification systems by plants, etc.

Unfortunately, these systems are very costly, difficult to implement, particularly in existing buildings, requiring complex maintenance which is in itself costly, and generates other pollutants, such as with filters. For systems with plants, there are also problems of renewal and maintenance of plants, which are complicated and require much labour and are difficult to automate.

There is therefore a real need for a system that can address these shortcomings, disadvantages and obstacles of the prior art.

### DESCRIPTION OF THE INVENTION

The present invention is specifically designed to meet the aforementioned needs and disadvantages of the prior art. The present invention more particularly relates to a device comprising:

a container for cultivating algae and/or microorganisms in an aqueous medium,

supply means for the algae and/or microorganism culture,

means for injecting an effluent into said algae and/or microorganism culture, with the said effluent coming from a building,

means for adjusting the temperature of the algae culture,

means of connection to the building from which the effluent comes from, to said means of injection, with the latter's function being to conduct the effluent from the building right up to the means of injection and,

optionally a lighting system for promoting the growth of algae and/or microorganism culture.

The present invention is also intended to show how this device can be used for treating an effluent and/or produce a biomass.

According to the invention, “building” refers to any structure used to house people, animals or things. It can be, for example, a construction, an industrial building and/or offices and/or houses and/or agricultural building, for example a house, a building, a thermal plant and/or an underground engineering structure, for example an underground infrastructure for vehicle and/or railway traffic, for example, a motorway tunnel, an underground railway tunnel, a car park, a tunnel, an underground road network, an area under slabs, a cave or grotto converted into a human or animal or culture habitat, or to be used for industrial or storage purposes.

According to the invention, the effluent can be, for example, a gas effluent or a liquid effluent.

According to the invention, a “liquid effluent” refers to one effluent only or a mixture of liquid effluents. It may be, for example any polluted liquid and/or solution from a building. The liquid effluent can be polluted by human occupation. It may be, for example, wastewater from toilets, a liquid containing a contaminant such as a metal, for example lead, nickel, a pollutant, such as nitrates and salts.

The present invention, particularly allows the re-treatment of polluted liquid effluents, such as those mentioned above, for example, effluents with metals, salts, chemical compounds also, and other pollutants that may be, for example, evacuated from buildings.

According to the invention, the treatment of the liquid effluent or the mixture of liquid effluents may consist of, for example, eliminating, in other words, extracting from the liquid effluent at least one pollutant, a contaminant such as a metal, for example lead, nickel, nitrates, salts and any pollutants. It may, for example, consist of desalting a liquid effluent and/or renewing the treated liquid. The treatment can be based on the algae and/or microorganism chosen. According to the invention, the algae and/or microorganism may be chosen depending on the desired treatment of the effluent to be treated.

In this document “gas effluent” refers to a single gas effluent or a mixture of gas effluents. It may be any foul air from a building or any mixture of foul air from a building. The gas effluent may be foul air caused by human occupation, vehicle traffic in a building, such as a car park or several car parks, or building surroundings, such as tunnels, underground road networks, areas under slabs, industrial productions, air from heating sources of buildings, especially gas oil and gas. According to the invention, a gas effluent may be a gas comprising for example CO₂, Nitrogen dioxide or foul air due to human occupation, vehicle traffic in the building (car parks), railway traffic, or building surroundings (tunnels, underground road networks, areas under slabs), industrial productions.

The present invention especially allows the re-treatment of polluted CO₂ and Nitrogen dioxide, carbon monoxide gas effluents, and other pollutants caused by humans, and are evacuated from buildings, especially those mentioned above, in particular by ventilation plants and other examples below.

According to the invention, the treatment of the effluent gas may consist, for example, of eliminating, in other words, extracting the gas effluent from the CO₂ and/or NO₂. Advantageously, the algae convert the CO₂ and/or NO₂ into oxygen that can, for example renew the atmosphere of the building or be evacuated. They may be gases other than those mentioned above, and may also consist of particles and dust found in these gases. The treatment can be a function of the algae and/or microorganism chosen. Similarly, the algae and/or microorganism can be chosen according to the effluent to be treated.

According to the invention, the means of injecting the effluent into the algae and/or microorganism culture may be connected to a means of recovering the effluent to be treated in order to recover the effluent(s) from one or more buildings and/or from one or more underground engineering works, such as underground infrastructure for vehicle and railway traffic, for example, a motorway tunnel, an underground train tunnel to inject in the algae culture. Advantageously, The present invention allows the treatment of foul air or the contaminated liquid of a building by connecting the aforementioned device used in The present invention to the building.

According to the invention, the means of recovering the said effluent can be chosen from the group consisting of a blower, a suction pump, a ventilation circuit, an air conditioning system, an air filtration system of a building. Any means of recovering an effluent to be treated can be used. This recovery means can be for example a pipeline for transporting the effluent(s), for example, from the building or from several buildings, for example, from one or more thermal plant(s) or any other building like those mentioned above. Of course, this recovery means is connected to the means of injection in order to bring the effluent into contact with the algae and/or microorganisms which are being cultured, the said algae and/or microorganisms designed to metabolize the pollutants and/or undesirable elements in the effluent, such as gas elements, for example CO₂ and or NO₂ to eliminate them.

According to the invention, the container for cultivating the algae and/or microorganism may be any container known to those skilled in the art. It may be, for example, in a form chosen from the group consisting of a pipe, a cylinder, a flat pipe, a pipe corrugated along its length and width, a hollow panel, a sphere, a cube, a parallelepiped rectangle, a spiral, a parallelepiped rectangle with rounded edges, a hollow shape with no sharp edges, of a sachet. In the vocabulary from the field of architecture, the term “pipe” also covers all these possible structures, once they are hollow. Preferably, according to the invention, the hollow shape has no sharp edges. Also, in this document, “pipe” refers to any form of container which can contain an algae and/or microorganism culture, including a pipe or a hollow panel. For example, it may be an extremely tiny sachet, for example an ethylene tetrafluoroethylene (ETFE) sachet; glass profile, for example, shaped as a parallelepiped, preferably with rounded edges, a hollow panel in glass profile preferably to culture the algae and/or microorganisms. This container indeed forms a reactor in which the algae and/or microorganism are cultivated. Thus, any shape suitable for the culture of algae can be used.

Preferably, the shape of the container used for cultivating the algae and/or microorganisms is a hollow parallelepiped rectangle with rounded edges, such as a hollow panel with internal and possibly external edges, which are rounded, or a pipe. Advantageously, the culture device is without sharp edges. Indeed, the absence of edges prevents the accumulation and/or fouling of algae and/or microorganisms which is observed in containers with sharp edges, at the level of the hollows formed by these edges.

According to the invention, the container should be preferably a container transparent to light. It can be, for example, a glass profile container, a polycarbonate or plexiglass pipe. It is of course the inner wall of the container. This is particularly preferred when the algae or the microorganism being cultivated needs light to grow and/or to treat the effluent, and that the light used is natural light.

According to the invention, the thickness of the container, that is its walls, can be between 5 cm and 60 cm, preferably between 15 and 20 cm. It may in fact be of any thickness that ensures the firmness of the container when it is filled with the culture medium and the algae and/or microorganisms. Those skilled in the art could easily determine this thickness.

According to the invention, the height of the container may be, for example, between 1 and 10 m, preferably between 2 and 8.5 m. In fact, any height can be used, provided it is constructible.

According to the invention, when the container is horizontal, the length of the container may be, for example equal to the length of the building and/or to its width, for example 100 metres.

Advantageously, when the container is horizontal, it can be placed, for example, as a coil or spiral around and/or on the building.

When the container is horizontal or inclined, it may be placed on the roof of the building. The roof slope can control the inclination of the container.

According to the invention, the container may include a reinforcement in which a light and/or back-light can be placed or lodged. For example, the container can be shaped, transversely, as a kidney bowl, with the light being lodged in the hollow of the kidney bowl. Many hollows or undulations may be envisaged to accommodate an artificial lighting system thereby providing the cultivated algae and/or microorganisms with the light needed for their growth and/or the treatment of the effluent. It may also be an artificial lighting system.

According to the invention, when the culture container is a pipe or a hollow panel, the pipe or hollow panel is also, and for the same reasons as those mentioned above, transparent to light, since the algae and microorganism culture are sensitive to this. This advantageously allows the algae and/or microorganism to benefit from natural and/or artificial light for its metabolism, in particular photosynthesis, especially in a bid to treat the effluent. As mentioned above, it can be, for example, a pipe or glass panel, a pipe or a polycarbonate or plexiglass panel, or any other material suitable for carrying out The present invention.

According to the invention, when it is a pipe, the outer diameter of the pipe can be, for example, between 20 and 100 cm, preferably between 40 and 80 cm. The thickness can be, for example, those mentioned above.

According to the invention, the height of the pipes can be between 1 and 10 m, for example, preferably between 2 and 8.5 m. The same remarks as those above on the container in general are applicable for the height.

According to the invention, the container, such as, the pipe or panel can, for example, be multilayered. It may include, for example, from the outside to the inside, concentrically, an outer layer, a middle layer and an inner layer, and further comprise a lighting or back-lighting system. The back-lighting lights the culture, for the reasons stated above, for example, when the environment does not provide enough light or the user wishes to stimulate the algae and/or microorganism culture.

Layer refers to the space created between two containers, for example, concentric pipes or panels, that is, placed within each other at the axis of containers, for example pipes or panels, which are parallel and leave a space between the containers, such as pipes or panels. Concentric refers to containers, for example pipes or panels. Concentric refers to one or more containers, for example pipe(s) or panel(s) placed in one or more other pipe(s).

The layer is therefore differentiated by the walls of the containers, such as pipes and/or panels. The containers may be similar or different in their constitution that is, in their shape and materials used. The distance between the concentric containers creates a space defined by the walls of the containers. This space depends on the diameter of each of the containers placed concentrically, preferably lengthways.

Preferably, the surface of the culture container in contact with the culture medium is a surface which inhibits or prevents any fouling, especially of algae and/or microorganisms, on the surface. It can be, for example, a surface previously treated with an antifouling chemical product.

The container may be for example a vertical, horizontal or inclined container. For example, the container can be inclined between 0 and 90°. Preferably, the container should be a vertical or horizontal container. It can be, for example, pipes or hollow panels or any other shapes mentioned above, vertical or not, for example, the inclination of the container, for example pipes, or others, can be vertical or horizontal, for example, between 0 and 90°.

According to The present invention, lighting system promoting the algae and microorganism culture refers to natural lighting, such as daylight or artificial light, for example emitted by a lighting source to reproduce daylight or a wavelength sufficient for the algae and/or microorganism culture.

According to the invention, the lighting source can be a source independent of the algae and/or microorganism culture, coming to add or replace natural lighting from the sun. According to the invention, the lighting can be done, for example, through one or more fluorescent light tube(s), light emitting diodes (LED), through one or more quartz halogen lamps. Preferably, the lighting can be done by one or more fluorescent light tubes, light emitting diodes, through one or more quartz halogen lamps whose light wavelengths are chosen between 430 and 660 nm, preferably equal to 430 nm or 660 nm. The lighting can be decorative and/or depend on the algae and/or microorganism and its needs for its growth and/or the treatment of the effluent.

The lighting source may be placed in a space created by the concentric arrangement of containers, for example, pipes and/or the hollow panels. It may also be placed and/or attached to another surface, for example a frontage of a building and/or come from the building itself. For example, when two containers are used, for example, pipes or panels, placed concentrically, one on the outside and the other placed on the inside, the back-light can be placed in the inner container or in the space created between the outer container and the inner container, in order to protect the lighting of the culture medium.

According to the invention, the algae can be chosen, for example, from the group consisting of Chlorophyceae, Chlorella, Parietochloris incisa, Diatoms Amphora sp., Nitzchia sp., Chaetoceros sp., Chrysophyceae (golden brown algae). In fact, advantageously, according to the invention, any type of algae is suitable, once it can be cultivated and can treat an effluent for the purposes of The present invention. Advantageously, it may be one or a mixture of micro algae which can produce bio-diesel.

According to the invention, the microorganism can be chosen from, for example, bacteria, yeasts, fungi. Advantageously, according to the invention, any type of microorganism can be used, once it can be cultivated and can treat an effluent for the purposes of The present invention. Preferably, the microorganism should be a bacterium. Preferably, the bacteria should be a cynobacteria (blue green algae). Preferably, the cynobacteria should be chosen from the group consisting of Spirulina platensis, Chroococcales Chamaesiphon, Chroococcales Gloeabacter, Chroococcales Synechococcus, Chroococcales Glocothece, Chroococcales Cyanothece, Chroococcales Gloecocapsa, Chroococcales Synechoexstis, Pleurocapsales Dermocarpa, Pleurocapsales Xenococccus, Pleurocapsales Dermocarpella, Pleurocapsales Myxosarcina, Pleurocapsales Chroococcidiopsis, Oscillatoriales Spirulina, Oscillatoriales Arthrospira, Oscillatoriales Oscillatoria, Oscillatoriales Lyngbya, Oscillatoriales Pseudanabaena, Oscillatoriales Starria, Oscillatoriales Crinalium, Oscillatoriales Microcoleus, Nostocales Anabaena, Nostocales Aphanizomenon, Nostocales Nodularia, Nostocales Cylindrospermum, Nostocales, Nostocales Scyytonema, Nostocales Calothrix, Stigonematales Chlorogloecopsis, Stigonematales Fischerella, Stigonematales Stigonema, Stigonematales Geitleria, Plochloraceae Prochloron.

The cultivation of algae and/or microorganisms may be achieved using any means appropriate known by those skilled in the art. According to the invention, the culture medium may be chosen according to the alga or algae to facilitate optimal cultivation preferably, and above all optimal metabolism for the treatment of an effluent, for example, a gas effluent. The algae may be stressed during cultivation so as to increase their efficacy in treating a gas or liquid effluent. According to the invention, the culture medium may be chosen according to the microorganism or microorganisms to facilitate optimal cultivation preferably, and above all optimal metabolism for the treatment of an effluent, for example, a gas effluent. The microorganisms may be stressed during cultivation so as to increase their efficacy in treating a gas or liquid effluent.

Numerous culture milieu are accessible on the internet and in specialised works. According to the invention, cultures in an aqueous medium are favoured. Stress may be caused, for example, by means of chemical molecules. Those skilled in the art are acquainted with these techniques and molecules.

According to the invention, the supply means for the algae and/or microorganism culture may consist of, for example, an automatic pump, means for regulating the supply of the algae and a supply reservoir. Any other appropriate means to ensure the cultivation of the algae may be used. All these means are those traditionally used by those skilled in the art to ensure continuous cultivation of algae and/or microorganisms.

According to the invention, the means for adjusting the temperature of the algae and/or microorganism culture may consist of, for example, a thermostat or any other appropriate means for controlling the temperature and reaction in case of undesired variations in temperature.

According to the invention, the device of the invention may also include means for controlling the temperature around the cultivation container. These control means may be connected to means for heating and/or cooling.

According to the invention, the means for heating the algae and/or microorganism culture may be chosen, for example, from a group comprising of means for recovering heat from a building, means for recovering exterior heat, means for recovering solar heat, means for recovering calorific energy.

For example, means for recovering exterior heat may consist of a heat pump for example; means for recovering heat from a building may consist of, a double skin comprising of an exterior covering positioned in front of the frontage of a building.

According to the invention, means for cooling the algae or microorganism culture may be chosen, for example, from a group comprising of means for recovering cold from a building, means for recovering exterior cold, means of refrigeration, for instance, air conditioning. For example, means for recovering exterior cold may consist of a heat pump. Means for recovering exterior cold and/or from a building may consist of, for example, a double skin comprising an exterior covering positioned in front of a building frontage.

According to the invention, means for cooling and heating may be the same, for example, a double skin.

According to the invention, the exterior covering of the double skin or double wall may be transparent and may comprise of air vents, said double skin preferably being fixed and parallel to the building frontage. The exterior covering of the double skin may consist of, for example, a glass surface, an ETFE surface, a surface formed from woodwork and glazing, woodwork and steel, metal, stainless steel, galvanised steel, nylon or carbon mesh. The container for cultivating algae and/or microorganisms may, advantageously, be positioned between said building frontage and said exterior covering. The air vents allow ventilation of the space between the exterior covering of the double skin or double wall and the building frontage.

According to the invention, the exterior covering of the double skin is preferably watertight, preferably, it can limit the passage of air and/or currents of air from the exterior, particularly in winter, so as to protect the container for cultivating the algae from variations in temperature.

According to the invention, the double wall or double skin may, advantageously, form an air cavity around the building and/or confine the heat and/or cold emitted by the building, thus, the device is capable of using the heat and/or cold emitted by the building to heat and/or cool the algae and/or microorganism culture.

Advantageously, the double skin also allows for the storage of calories from the building, so as to improve the calorific inertia of a building and adjust the temperature for cultivation.

Advantageously, The present invention makes it possible to recover the heat and/or cold from a building to cultivate the algae and/or microorganisms and at the same time, thanks to the cultivation of algae and/or microorganisms, treat effluents generated, for example, by said building.

According to the invention, the device may also consist of means for recovering the biomass formed from cultivating algae.

According to the invention, said device may also comprise of a drainage system for the container for cultivating algae and/or microorganisms. This system may be able to clean the container and/or device for the present invention in its entirety. It may also allow for, if required, the recovery of the biomass formed so that it can be reused as indicated above.

According to the invention, said device may also consist of one or several means for controlling and regulating the cultivation of algae and/or microorganisms as follows: means for controlling the supply of the algae, means for controlling the injection of the effluent to be treated, means for controlling the temperature, means for controlling the pH, means for controlling the lighting system for the algae. These means may consist of, for example, those currently used for cultivating algae and/or microorganisms, and more generally microorganisms.

According to the invention, advantageously, said device may be operated by computer in order to facilitate the optimisation for cultivating algae and/or microorganisms and/or the treatment of the effluent. The computer may be, for example, connected to the thermostat on the container for cultivating algae and/or microorganisms and to different control means installed to make the device of the invention work.

According to the invention, the device of the invention may function continuously or intermittently. It may function, without interruption, day and night. The lighting system for the algae and/or microorganisms may, thus, be permanently maintained so as to keep the culture active for treating the effluent.

According to the invention, the frontage of the building may include, advantageously, a railing. The railing may permit, for example, individuals to move along the device, upkeep and maintenance of the device.

For example, the railing may be composed of metal, or glass. For example, it may be composed of metalwork and steel, metal, stainless steel, galvanised steel, nylon or carbon mesh, metal bars and/or metalwork and an ETFE membrane.

According to the invention, the device may further comprise a reinforced structure to support the container for cultivating algae and/or microorganisms, the container for cultivating algae and/or microorganisms may consist of, for example, a pipe for cultivating algae and microorganisms, said reinforcement may be fixed to the building. This reinforced structure may be fixed to the building, self-supporting or fixed to the exterior covering of the double skin. If the container for cultivating algae is positioned on a building frontage, this can support it.

According to the invention, said device may also comprise at least one passageway, said passageway may be used to support the container for cultivating algae and/or microorganisms and/or means of access to the container for cultivating algae and/or microorganisms for maintenance staff.

Thus, according to the invention, advantageously, one may form a genuine biological frontage for a building, which allows for not only the treatment of polluted air emitted into and by the building, insulation of the building, recovery of heat from the building for cultivating algae and/or microorganisms, but allow also producing a reusable biomass, notably as a biofuel, in the pharmaceutical domain and the food-processing domain. It may be, for example, for chemical compounds and proteins.

According to the invention, advantageously, the biomass which may be produced from cultivating algae and/or microorganisms may in effect be recovered and reused, notably as biofuel, a chemical compound or pharmaceutical compound.

The biomass may be able to produce an oleaginous microorganism.

According to the invention, the biomass may be able to be converted into coal or biopetroleum, using techniques known to those skilled in the art.

According to the invention, the biomass may be used directly to produce electricity. It may also be transported, through a pipe for example, to a building, such as a treatment plant. It may also be converted, for example, by oil pressing or pyrolysis treatment using techniques known to those skilled in the art to produce coal or biopetroleum.

According to the invention, advantageously, the device may be integrated into the structure of a building or added onto the building.

The present invention, thus, makes it possible for building frontages to produce primary biochemical energy obtained by photosynthesis, notably biofuels.

Advantageously, The present invention makes it possible to use climatic, chemical and structural opportunities to integrate a biochemical process inside or along the frontages, which allows building frontages to work on a state of symbiosis between the two systems used simultaneously, one recycling emissions from the other in order to produce the energy it needs.

The present invention, can advantageously, be implemented across all building surfaces, building coverings, frontages or roofs, underground works, for example, an underground structure for vehicle and railway traffic, such as a new or existing motorway tunnel or an underground railway tunnel. For example, the surface may be a poorly exploited section of the buildings such as the exterior covering or any other surface or surfaces of the building or an interior surface of the building. The surface chosen to position the device of the invention is preferentially a surface which profits from a large developed available surface, a high disposition, an incidental radiation, thermal emissions, from its infrastructure, and chemical provisions from the building. The surface may be for example, a concrete surface, a glazed surface, a surface composed from a waterproof complex and cladding or a photovoltaic surface. The optimal conditions are those for the cultivation of the chosen algae.

Limiting the surface coverage to the ground, The present invention may be located anywhere, even in an already populated urban environment. It may also be applied to existing building frontages during restructuring or renovation operations. Herein, it responds to the current office block restructuring campaigns to bring them into line with new environmental standards and requirements.

From the perspective of the building, the present invention may advantageously integrate recent evolutions of bioclimatic buildings. In comparison with traditional photovoltaic energy frontages, it has the advantage of being able to work in any direction.

The container for cultivating algae and/or microorganisms may be a bioreactor. It may be placed on a surface such as that previously defined. The bioreactor may, for example, be on and/or in front of a building frontage and may be served by exterior passageways.

the passageways are, advantageously, capable of protecting the cultivation of algae and/or microorganisms from solar overheating in summer,

vertical or horizontal bioreactors provide additional solar protection,

restriction of glazed surfaces for the exterior covering of the double skin can, advantageously, avoid overheating in summer and it is also compatible with the surface available for lighting the premises and leaves the device free,

the inertia of the building may further be improved by pipes and or hollow panels containing water on the frontage to regulate thermal overheating. These water pipes and/or hollow panels are able to regulate the temperature for cultivating algae and/or microorganisms, as well as participate in supplying the algae and/or microorganism culture.

The culture container itself may be considered as a water container and thus, also participate in the regulation of thermal overheating.

The device of the present invention may also comprise, for example, means to improve the thermal protection of the bioreactors.

The present invention has also for object a container for cultivating algae and/or microorganisms or a bioreactor such as that previously defined; this device may be integrated in a ventilated double skin frontage. This double frontage is the double skin defined below.

For example, the bioreactor may be contained between two surfaces. For example, a surface may be an “interior surface”, such as a surface of the building, and an exterior surface, in other words, a surface other than the interior surface such as the exterior covering, these two surfaces define a double skin.

The advantages of the double skin are as follows:

the same as those of the invention, as well as:

a greenhouse effect with free calorific gains in winter,

a cool and ventilated space in summer by opening up of the double skin,

direct recovery of energy dispelled by the covering of the building which is recovered by the process,

optimised use of the thermal inertia from the water pipes which makes it possible to regulate this intermediary space by storing the surplus heat and releasing it during reduced solar energy supply.

The exterior covering of the double skin or exterior surface may be composed of, for example:

opening or sliding or fixed windows,

horizontal, vertical or oblique glass jalousies allowing light to pass,

ethylene tetrafluoroethylene (ETFE) inflatable surfaces with integrated ventilation,

surface composites or any other material or system ensuring transparency and/or ventilation sufficient for the requirements of the building.

The double wall or double skin may, advantageously, produces a parietodynamic frontage allowing recovery of surplus calories or energy which are reconverted into cold and/or heat, using a HP (heat pump) for example. In winter the system may be reversed with a reversible heat pump.

According to the invention, the container for cultivating algae and/or microorganisms may also be placed inside a building.

The building may be for example, a building designed to minimise its flow and energy expenditure.

The concept of mutualisation may be global and encompass the whole building.

For example, ventilations from frontage to frontage may permit pre-treatment or thermal recycling of air using the space of the double frontage and the inertia supplied by the water masses. Advantageously, in summer, for example, the double frontage or double skin may be a frontage that is more than 50% open: the shade from the installations and passageways may restrict the solar gains and thus, air conditioning. In winter, the double frontage may be a closed double skin allowing the formation of an insulating air cavity around the building.

One of the many objectives achieved through the present invention is to obtain a building that is:

as passive as possible, from the perspective of the storage of water, from the effect of solar protection and the insulating space of the double frontage,

energetically autonomous, indeed, over productive.

The present invention makes it possible, for example:

to rid pollution, since the bioreactor can convert carbon dioxide and nitrogen dioxide into oxygen and eliminate lead and nickel for example, from nitrates, and/or desalinise a liquid effluent,

grow oleaginous microorganisms to produce energy, from organic material such as proteins and chemical compounds.

The energy production from the biomass thus produced by the present invention is 130 times more effective, with an equal culture surface, than production of an equivalent biofuel from traditional farming, harvesting is done over a short cycle of 1 to 3 days for several months by field farming.

The present invention may also produce all kinds of interesting by-products, for example, proteins and components for organic material that can be used in the pharmaceutical sphere for instance.

The present invention can also produce raw energy materials at the place where they will even be consumed, and thus:

avoid any load losses linked to transport,

constantly recycle combustion waste.

It can also fully recycle CO₂ emissions linked to energy production.

Furthermore, it can produce energy from combustion waste up to when it is released into the atmosphere.

The present invention is able to optimise the production and productivity of bioreactors known prior art, for example, bioreactors from Biopetroleo BASF in Spain, those from Samash in France, Greenfuel, Shell/Petrosun and Valcent in the USA, Alguatech in Israel, Aquaflow bionomic corporation in New Zealand, whilst putting at the disposal of the former frontage surfaces, an infrastructure, and in the case of double frontages, a temperate space, prechilled or preheated freely by the double frontages and recycling of thermal losses and air emissions from the building.

The present invention also contributes to the thermal regulation of the building authorising double frontages with an agricultural vocation (not taking into account the net surface area) where the rule for defining the net surface area is prohibited from realising classic double frontages as they would then be taken into account in the calculation for the net surface area and would, therefore, unacceptably lower the surface performance of the net surface area/gross usable area ratio.

It also contributes to the thermal regulation of the building by introducing water masses into the double frontages creating high inertia capable of “smoothing out” the energy peaks resulting from climatic factors or factors linked to the nycthemeral rhythm of the building.

### BRIEF DESCRIPTION OF THE FIGURES

FIG. 1 shows an embodiment of the invention. Figure A shows a plan view of the device of the invention, in particular a frontage (F), the tubes T1 disposed between the frontage of the building (Bat) and a guardrail (GC). The tubes comprise a culture medium (M). FIG. 1B shows a front view of the frontage (F) and the guardrails (GC) fixed to the gangway (C) by bolting. Figure C shows a transverse section of the building on which the frontage of the building (F) is indicated with an alternation of glass panels (VI) and insulating complex (CI), the gangway (C) and the tubes T1 are fixed on the gangway (C) to the primary framework (O) by means of an angle bracket (A).

FIG. 2 shows an embodiment of the invention. FIG. 2A shows a plan view of the device of the invention, in particular a frontage (F), the tubes (T1) disposed between the frontage of the building (Bat) and the external envelope of the double skin (DP). The tubes comprise a culture medium (M). FIG. 2B shows a front view of the frontage (F) and glass panels (VE) of the external envelope of the double skin (DP) supported by external uprights (XE) and external crossmembers (YE). FIG. 2c shows a transverse section of the building on which the frontage of the building (F) is indicated with an alternation of glass panels (VI) and insulating complex (CI), the gangway (C), the tubes (T1) are fixed on a gangway (C) to the primary structure (0) by means of an angle bracket (A). The tubes are positioned behind the double skin (DP).

FIG. 3 shows an embodiment of the invention. FIG. 3A shows a plan view of the device of the invention, in particular a frontage (F), the tubes T1 disposed between the frontage of the building (Bat) and the external envelope consisting of an ethylene tetrafluorethylene membrane. The tubes comprise a culture medium (M). FIG. 3B shows a front view of the frontage (F) and of the ethylene tetrafluorethylene membrane (ME) supported by external uprights (XE). FIG. 3C shows a transverse section of the building on which the frontage of the building (F) is indicated with an alternation of glass panels (VI) and insulating complex (CI), the gangway (C), the tubes (T1) are fixed on the gangway (C) to the primary structure (0) by means of an angle bracket.

FIG. 4 shows an embodiment of the invention. Figure A shows a plan view of the device of the invention, in particular a frontage (F), the tubes T1 disposed between the frontage of the building (Bat) and the external envelope of the double skin (DP) and inside the building. The tubes comprise a culture medium (M). FIG. 4B a front view of the frontage (F) and the glass panels (VE) of the external envelope of the double skin (DP), supported by external uprights (XE) and crossmembers (TE). Figure C shows a transverse section of the building on which the frontage of the building (F) and the roof (TO) fixed to the building (Bat) by means of the primary structure (O) are indicated, the tubes (T1) are fixed to the deck (D) by means of an angle bracket (A). The tubes are positioned behind the double skin (DP) and under the roof (TO) and outside the building (Bat).

FIG. 5 shows an embodiment of the invention. FIG. 5A shows a plan view of the device of the invention, in particular a frontage (F), the hollow panels (T2) disposed between the frontage of the building (Bat) and the external envelope of the double skin (DP). The panels comprise a culture medium (M). FIG. 5B shows a front view of the frontage (F) and glass panels (VE) of the external envelope of the double skin (DP) supported by external uprights (XE). FIG. 5C shows a transverse section of the building on which the frontage of the building (F) is indicated with an alternation of glass panels (VI) and insulating complex (CI), the gangway, the hollow panels T2 are fixed on the gangway to the primary structure (O) by means of an angle bracket. The hollow panels are positioned behind the double skin (DP) at different angles.

FIG. 6 shows an embodiment of the invention. FIG. 6A shows a plan view of the device of the invention, in particular a frontage (F), the tubes (T3) are disposed between the frontage of the building (Bat) and the external envelope of the double skin (DP). The tubes comprise a culture medium (M). FIG. 6B shows a front view of the frontage (F) and glass panels (VE) of the external envelope of the double skin (DP) supported by external uprights (XE). FIG. 6C shows a transverse section of the building on which the frontage of the building (F) is indicated with an alternation of glass panels (VI) and insulating complex (CI), the gangway (C), the tubes T3 are fixed on the gangway (C) to the primary structure (O) by means of an angle bracket. The tubes are positioned behind the double skin (DP). They consist of an external tube (T3ext) and an internal tube (T3int).

FIG. 7 shows an embodiment of the invention. FIG. 7A shows a plan view of the device of the invention, it is identical to FIG. 1A. FIG. 7B shows a front view, it is identical to FIG. 1B. FIG. 7C shows a transverse section of the building, it is identical to FIG. 1C. FIG. 7D shows a tube T3 positioned between the frontage F and a guardrail (GC). FIG. 7E shows a tube T3 positioned between the frontage F and an ethylene tetrafluoroethylene membrane (ME). FIG. 7F shows a tube T3 positioned between the frontage F and an external envelope (PP) consisting of glass shutters. FIG. 7G shows a tube T3 positioned between the frontage F and an external envelope (DP).

FIG. 8 shows an embodiment of the invention. FIG. 8A shows a plan view of the device of the invention, in particular a frontage (F), the tubes (T4) disposed between the frontage of the building (Bat) and the external envelope of the double skin (DP). The tubes comprise a culture medium (M) and are positioned against the double skin (DP). FIG. 8B shows a front view of the frontage (F) and the glass panels (VE) of the external envelope of the double skin (DP) supported by external uprights (XE). FIG. 8C shows a transverse section of the building on which the frontage of the building (F) is indicated, the gangway (C), the tubes T4 are fixed on a gangway (C) to the primary structure (O) by means of an angle bracket (A). The tubes are positioned behind the double skin (DP). FIG. 8D shows a tube (D4) positioned between the double skin (DP) and the frontage (F) of the building.

FIG. 9 shows an embodiment of the invention. FIG. 9A shows a plan view of the device of the invention, in particular a frontage (F), the tubes (T4) disposed between the frontage of the building (Bat) and the external envelope of the double skin (DP). The tubes comprise a culture medium (M) and are positioned against the frontage (F). FIG. 9B is identical to FIG. 8B. FIG. 9C shows a transverse section of the building on which the frontage of the building (F) is indicated, the tubes (T4) are fixed on a gangway (C) to the primary structure (O) by means of an angle bracket (A). The tubes are positioned behind the double skin (DP). FIG. 9D shows a tube (T4) positioned between the frontage (F) and a guardrail (GC). FIG. 9G shows a tube (T4) positioned between the frontage (F) and an ethylene tetrafluoroethylene membrane (ME). FIG. 9E shows a tube (T4) positioned between the frontage (F) and an external envelope (PP) consisting of glass shutters. FIG. 9F shows a tube T4 positioned between the frontage (F) and an external envelope (DP).

FIG. 10A is a photograph showing vertical tubes of alga cultures (T3) with a culture support (SC). FIG. 10B shows two transverse sections of two multilayer tubes with an internal tube (Tint), an intermediate tube (Ti) and an external tube (Text). FIG. 10C shows different embodiments of the device, namely different inclinations, and FIG. 10D shows the different heights of the tubes and FIG. 10E shows a perspective view of a tube (T3) comprising an internal tube (T3int) and an external tube (T3ext), concentric with each other.

FIG. 11A is a schematic representation of a building (Bat) seen from above with its frontage (F) and showing the external envelope of the double skin (DP). FIG. 11B shows the exploded axonometry of a building with tubes (T1) fixed in front of the frontage (F) and behind the external envelope of the double skin (DP). The building is equipped with vertical tubes (T1) supported by the roof and installed underneath a roof (TO).

FIG. 12 is a general diagram showing an example of the treatment of gaseous effluent with the present invention.

FIG. 13 shows different example embodiments of the invention.

FIG. 13A shows different double frontages and guardrails, namely one in which the double frontage is a double glass frontage (VE) with metal uprights (XE) and gaskets (J), a double frontage with metal uprights (XE) and an ETFE membrane (ME). A glass guardrail (V1) with metal uprights (M1) and gaskets (J), a guardrail formed by metal bars.

FIG. 13B shows different concrete or metal gangways. FIG. 13C shows different building frontages, namely a frontage consisting of joinery and glass, a concrete wall or a sealed complex and cladding.

Other advantages can also be clear to a person skilled in the art in the light of the accompanying figures, given by way of illustration.

### EXAMPLES

### Example 1

### Device According to the Invention Disposed on a Building Frontage

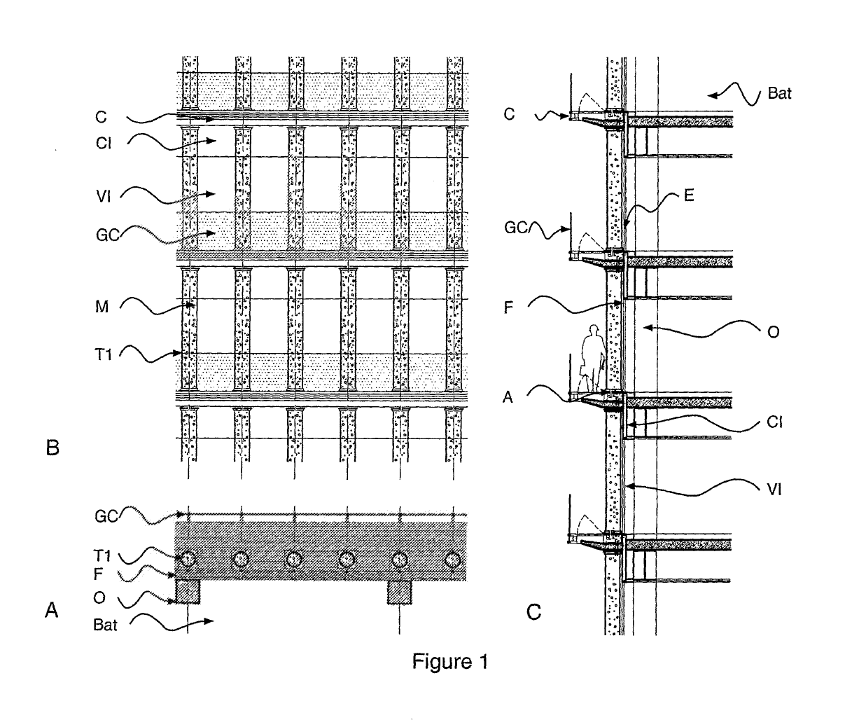

A description is given here of an embodiment of the invention. The device used is shown schematically in FIG. 1.

The alga culture used is Chlorella. It is cultivated in an aqueous culture medium, namely the Walnes medium, that is to say for 10 litres of medium 680 g of sodium nitrate (NaNO₃), 200 g of sodium dihydrogenophosphate, 400 g of sodium ethylenediamine tetracetic acid (Na₂EDTA), 20 g of boric acid (H₃BO₃), 40 ml of a 500 ml solution containing 30.5 g of potassium bromide (KBr), 6.5 g of strontium chloride (SrCl₂, 6H₂O), 0.25 g of aluminium chloride solution (AlCl₃, 6H₂O), 0.1 g of rubidium chloride (RbCl), 0.05 g of lithium chloride (LiCl, H₂O), 0.025 g of potassium iodide (Kl) and 800 ml of a 10 litre solution containing 213.2 g of ferric chloride hexahydrate (FeCl₃, 6H₂O), 15.0 g of manganese sulphate monohydrate (MnSO₄, H₂O), 2.5 g of zinc sulphate (ZnSO₄), 2.0 g of copper sulphate pentahydrate (CuSO₄, 5H₂O), 0.26 g of cobalt sulphate heptahydrate (CuSO₄, 7H₂O), 0.14 g of sodium molybdate dihydrate (Na₂MO₄, 2H₂O) and 0.10 g of sodium fluoride (NaF).

The alga culture is effected in the above medium at a temperature of 20° C. The pH of the medium is 7.3.

The means of feeding the alga culture is the plate itself, which makes it possible both to cultivate the alga and to feed it. It also comprises a means of injecting the gaseous effluent, which consists of an orifice situated in the moulded part, forming the base of the tube (T1), with a diameter of 2 cm, onto which there is screwed a tube for routing the gaseous effluent into the alga culture.

The tube also comprises at its base a second orifice with a diameter of 7 cm onto which there is screwed via a flexible polyethylene tube with a diameter of 5 cm to a Lambda Preciflow (registered trademark) feed pump.

The plate also comprises a third orifice with a diameter of 7 cm situated in the moulded base of the plate connected to a Lambda Preciflow (registered trademark) pump allowing aspiration of the culture medium.

The temperature of the alga culture is controlled via a temperature sensor marketed by the company Testo positioned in the moulded base of the plate and immersed in the culture medium.

A pH meter sensor from Testo is immersed in the culture medium, thus enabling the pH to be monitored.

A temperature sensor from the company Prosensor is immersed in the culture medium.

A sensor measuring the concentration of CO₂ in the medium is present in the culture medium.

These sensors are connected to a monitoring device for monitoring the change in pH, the CO₂ concentration and the temperature of the medium.

Every 24 hours, 50% of the volume of the culture medium is pumped and stored in a reservoir, the pumped medium comprises algae and corresponds to the biomass. An identical volume of fresh medium is simultaneously injected into the culture container. The culture medium is thus renewed so as to ensure optimum growth of the algae and therefore optimum treatment of the effluent.

The culture container is a tube (T1) shown in FIG. 1. The tube (T1) consists of a cylindrically shaped profiled glass.

The glass has a thickness of 5 cm and the height of the tube is 3.2 m. The diameter of the tube is 40 cm. It comprises a moulded rectangular piece 50 cm wide and 50 cm long. This piece holds the glass by strapping with a moulded piece at the bottom and top, that is to say at height of 3 cm and 320 cm respectively. The volume of the medium contained in this plate is approximately 400 litres.

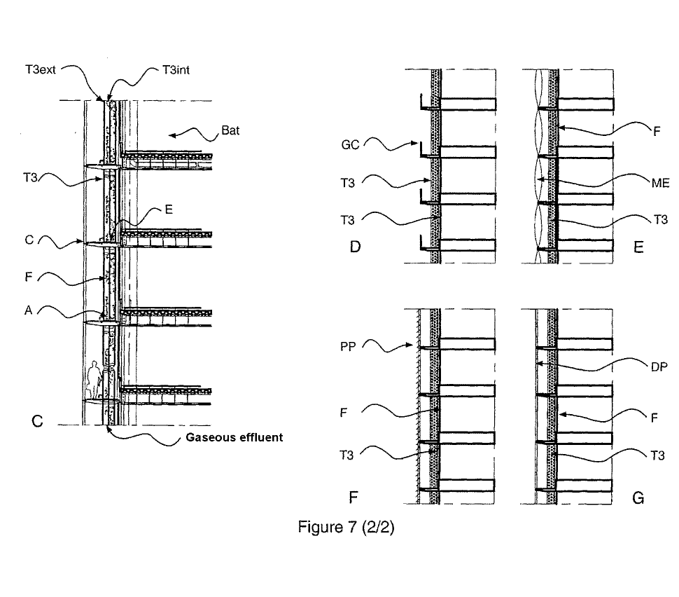

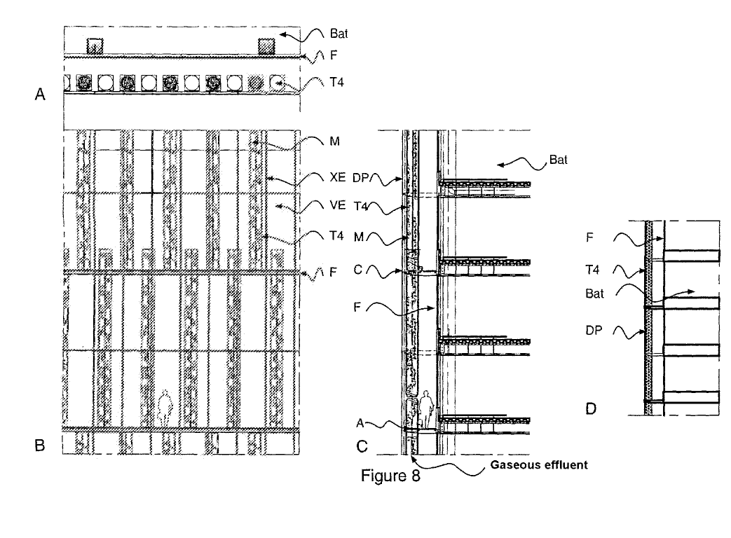

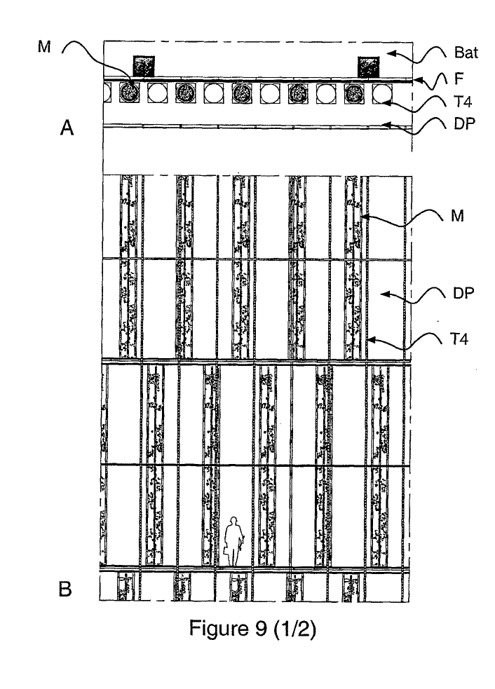

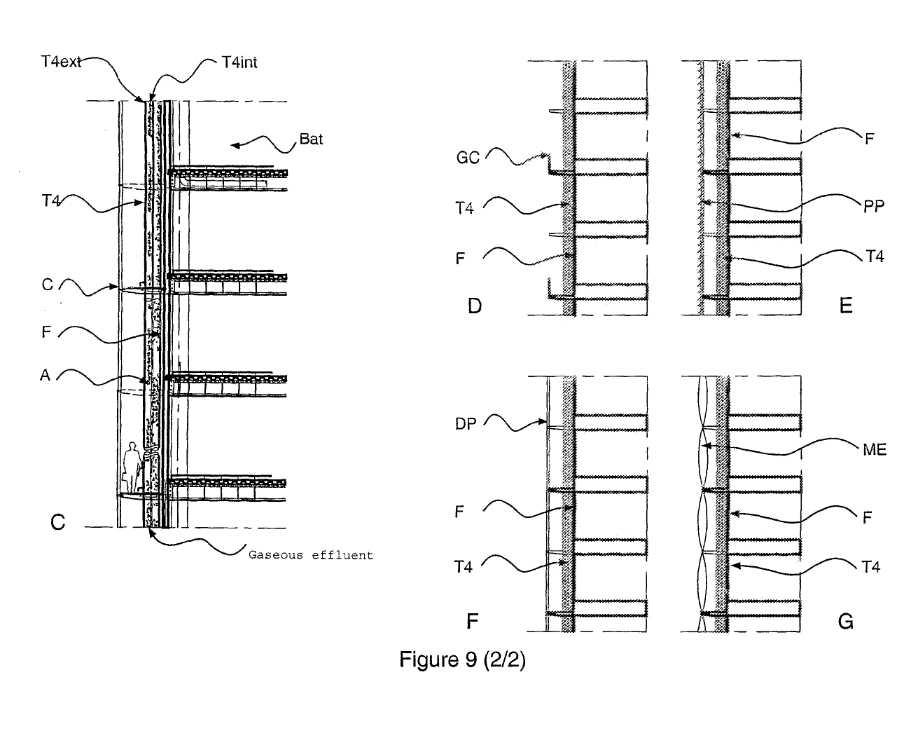

The tubes (T1) are designed in Saint Gobain laminated glass with an internal and external surface treatment by application of the product A.X.P.1 marketed by the company Matt Chem in order to avoid the attachment, for example, of algae (antifouling). The tube is held between two moulded parts made from a neutral material, that is to say stainless steel. The plate is placed on a PRS welded recomposed profile in the form of angle bracket shown in FIG. 7C (A). It comprises a broad hole, that is to say 20 cm in diameter vertically in line with the tube to enable the fluids to pass. At the top part, a steel element 5 cm thick and 3 cm wide encircles the plate in order to prevent it from spilling. The angle bracket (A) is fixed to the primary framework (O) by through steel rods.

The gangway (C) is a fabricated concrete console fixed in the primary framework (O) by through rods.

The gangway (C) enables the plates to be held from the outside. It is sufficiently wide, that is to say a minimum of 70 cm, to enable the tube to be installed and a person responsible for maintenance and replacement of the tube where necessary to pass.

The tube (T1) can be retracted from the angle bracket (A) by means of a maintenance carriage and a rail at the top part that serves as a guide for it.

In particular, the tube (T1) is disposed parallel, against the frontage (F) of the building. This frontage is a curtain or wall with rebating on four sides with an alternation of Saint Gobain glazing (VI) and opaque insulating complex (CI) formed with a thickness of insulation of 10 cm wedged between two aluminium panels, in front of the slab, in order to ensure fire safety for the building. The framework carrying the curtain-wall frontages is produced from square steel profiles fixed by bolting to the concrete primary framework of the building. The external cladding of the uprights and crossmembers is produced in a tubular aluminium profile with a cross section of 5 cm wide by 3 cm deep. The insulation between the outside and inside is provided by an intermediate seal of ethylene propylene diene monomer (EPDM) sold by the company Alder, 9 mm wide. The characteristics of the glazing brackets (VI) can be summarised as follows: 10 mm of external toughened safety glass—14 mm vacuum—12 mm internal laminated safety glass, the glasses coming from the company Saint Gobain.

Solar protection of these frontages is provided by the concrete gangways that fulfil the function of an external sun break. The vertical tubes also fulfil a role of protection from incident solar radiation.

The glass plate is placed on the angle bracket (A) in front of the frontage (F). The gangway (C) is sized so as to enable a person to pass and stand in front of the tube in order to provide maintenance and replacement thereof where necessary. The width of the passage is a minimum of 70 cm.

In order to protect the passage of a person providing the maintenance of the tubes from the outside, guardrails (GC) at a minimum height of 1 m are fixed to the gangway by means of a brace. A handrail formed by a flat iron rail 50 mm by 12 mm is fixed to the upright by a brace.

The pump supplying the culture medium is situated in the basement of the building with the technical rooms.

The pump pumping the culture medium is situated in the basements of the building with the technical rooms.

Electrochemiluminescent diodes emitting a wavelength of 630 nm are fixed by bolting to a steel plate at the rear of the tubes on the building frontage. Lighting of these diodes is controlled by a timer affording lighting at night.

An extractor is disposed on the pipe discharging the gaseous effluent from the building (Bat). This is a fan enabling the effluent to be routed in a duct 2 cm in diameter to the culture container. This fan is connected to the monitoring device thus making it possible to increase or decrease the flow of effluent according to the concentration of CO₂ in the culture medium.

Thus the CO₂ is recovered from the building in order to inject it into the device of the invention.

The algae are cultured in the aforesaid medium and the biomass recovered by pumping the medium into the storage reservoir.

Surprisingly, the inventors find that the arrangement of the plates or of a culture container in front of the building frontage makes it possible to insulate the latter from the external air and to increase its energy efficiency.

The addition of CO₂ and NO₂ by gaseous effluent into the culture medium containing the algae in the presence of sun and/or under lighting with the diodes makes it possible to recycle the CO₂ and NO₂ while producing organic matter and emitting oxygen through the algae. This recycling is done naturally by the algae via the biosynthesis process. This effluent also heats the algae culture medium.

Surprisingly, the inventors find that the mean temperature of the culture is more stable when the container is situated in front of the building frontage. Thus the production and purification yields of the gaseous effluent are also superior.

This example therefore clearly demonstrates that the device of the invention makes it possible both to insulate the building but also to recycle the gaseous effluent issuing from the buildings treated by the device of the present invention.

### Example 2

### Device of the Invention Disposed on a Frontage of a Building Producing Biomass

In this example, the device described in Example 1 is used to form biomass.

In this case, an extractor is disposed on the pipe discharging the gaseous effluent into a thermal station adjacent to the building (Bat).

Thus the CO₂ is recovered from an external source of the building in order to inject it into the device of the invention.

The algae grow more rapidly with this addition of CO₂.

### Example 3

### Device According to the Invention Disposed on a Frontage of a Building with a Glass Double Skin

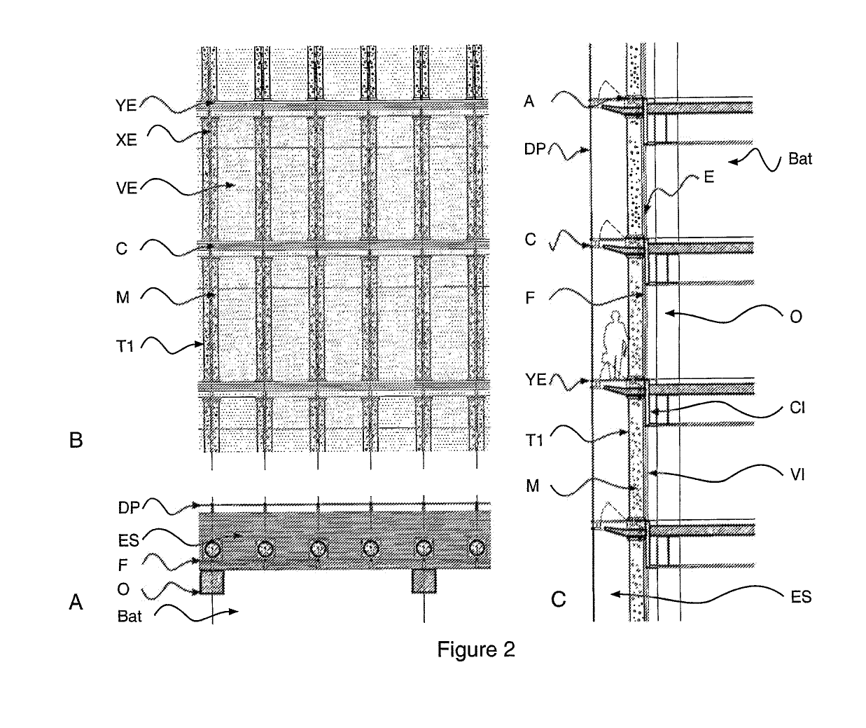

FIG. 2 shows the device of Example 1 disposed between the frontage of a building (F) that becomes the internal envelope and a glazed overfrontage, the latter forming the external envelope (DP).

FIG. 2A is a plan view, 2B is a front view and 2C a transverse section.

The algae, culture medium and culture conditions are identical to those in Example 1.

In this example, the culture container is a vertical tube (T1). This tube is a glass tube with a diameter of 40 cm. The size of the tubes is 3.20 m as shown in FIG. 2.

Electrochemiluminscent diodes (E) emitting a wavelength of 630 nm are fixed by bolting to the frontage of the building. The lighting of these diodes is controlled by a timer allowing lighting at night.

The external envelope (DP) is a curtain or wall with rebating on four sides. The steel bearing framework of the curtain wall frontages is produced from square profiles 20 cm deep and 5 cm wide made from steel, fixed by bolting to the prefabricated concrete gangway (C). The gangway is identical to the one in Example 1.

The external cladding of the uprights and crossmembers is produced in tubular profiles 5 cm wide by 3 cm deep in aluminium. The insulation between the outside and inside is provided by an ethylene propylene diene monomer (EPDM) intermediate seal sold by the company Alder, 9 mm wide. The characteristics of the glazing (V) can be summarised as follows: 10 mm of external toughened safety glass—14 mm vacuum—12 mm internal laminated safety glass, the glasses coming from the company Saint Gobain.

The glass tube (T1) is placed on an angle bracket (A) in front of the frontage (F) of the building. The angle bracket (A) is identical to the one in Example 1. The gangway (C) is sized to enable a person to pass and stand in front of the tube in order to maintain and replace it where necessary. The passage width is a minimum of 70 cm.

A person maintaining the tubes passes inside the double skin between the internal envelope and the glazed external envelope (DP).

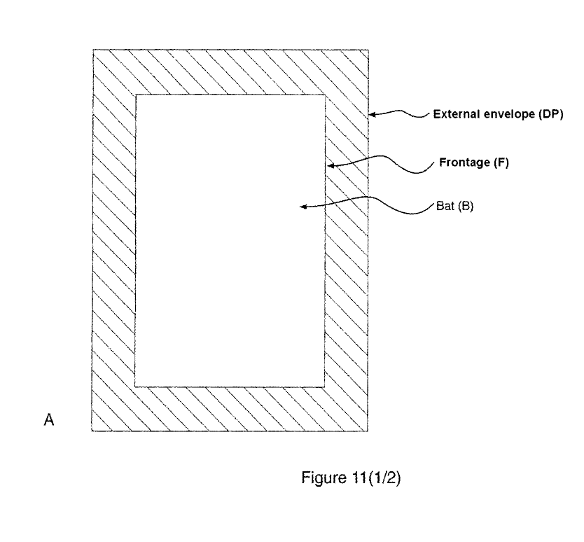

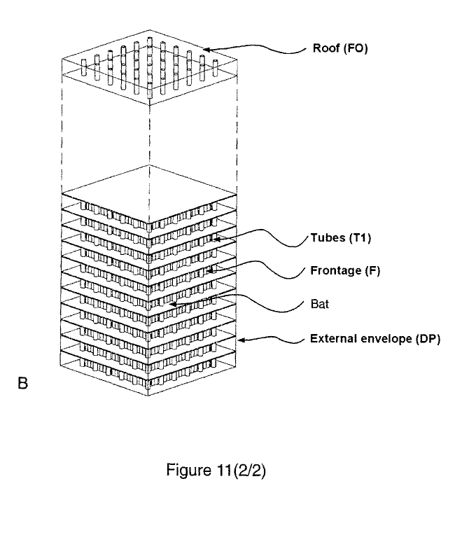

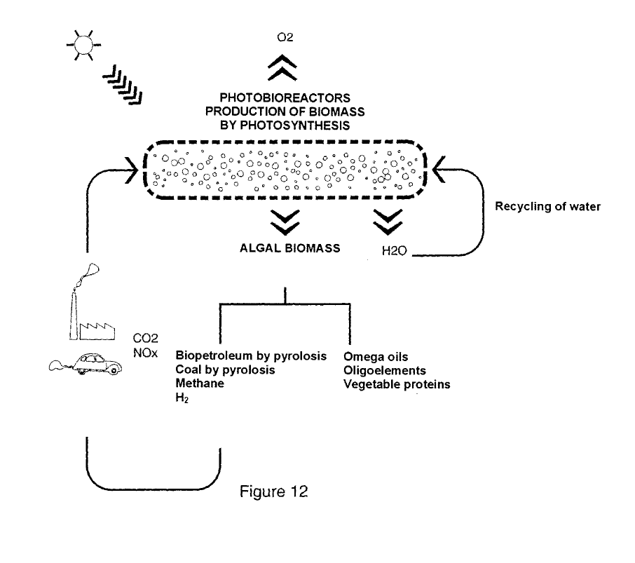

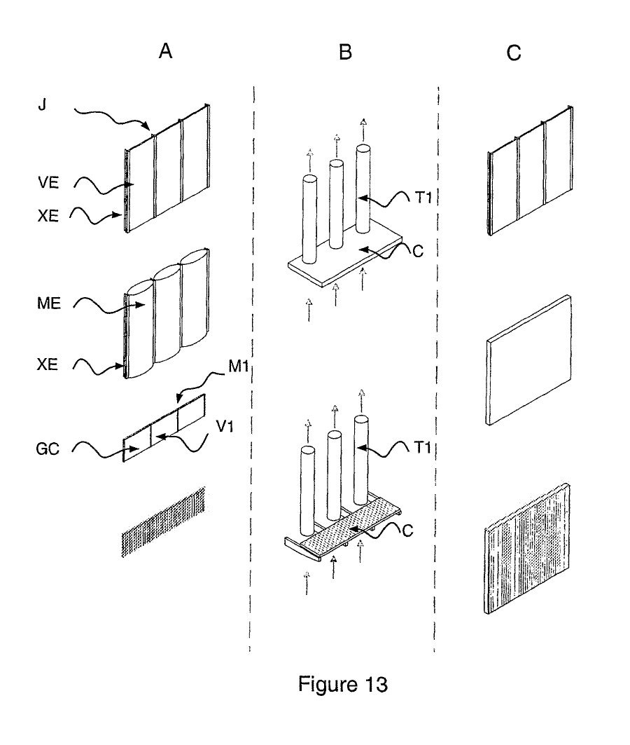

This example embodiment comprising a double skin is shown schematically in FIG. 11B, which shows a plan view of a building (Bat) with an external envelope (DB) and a space (ES) in which the device is present.

The double skin demonstrates a very good result in terms of thermal insulation since the building is ventilated with air being taken on the inside of the double skin, which itself remains at a temperature close to that of the internal spaces of the building.

It makes it possible to delimit a space in which the culture container is situated. The inventors therefore demonstrate surprisingly that this double skin also defines a space in which the temperature is regulated by the heat contribution of the building. This envelope also insulates the culture container from the external air and thus regularises the alga culture conditions.

The addition of CO₂ and NO₂ by gaseous effluent in the culture medium containing the algae in the presence of sun and/or under lighting with the diodes makes it possible to recycle the CO₂ and NO₂ whilst producing organic matter and emitting oxygen by the algae. This recycling is done naturally by the algae via the biosynthesis process.

The recovery of the biomass and the renewal of the culture medium is achieved as indicated in Example 1.

When this example is implemented, the inventors find surprisingly that the mean temperature of the culture is more stable when the container is situated between the frontage (F) of the building and the external envelope of the double skin (DP). The production yields of the biomass and purification of the gaseous effluent are also superior to full-field devices.

This example therefore clearly demonstrates that the present invention makes it possible both to isolate the building and also to recycle the gaseous effluent issuing from the buildings and treated by the device of the present invention.

### Example 3

### Device According to the Invention Disposed on a Frontage of a Building with an Ethylene Tetrafluoroethylene ETFE Membrane

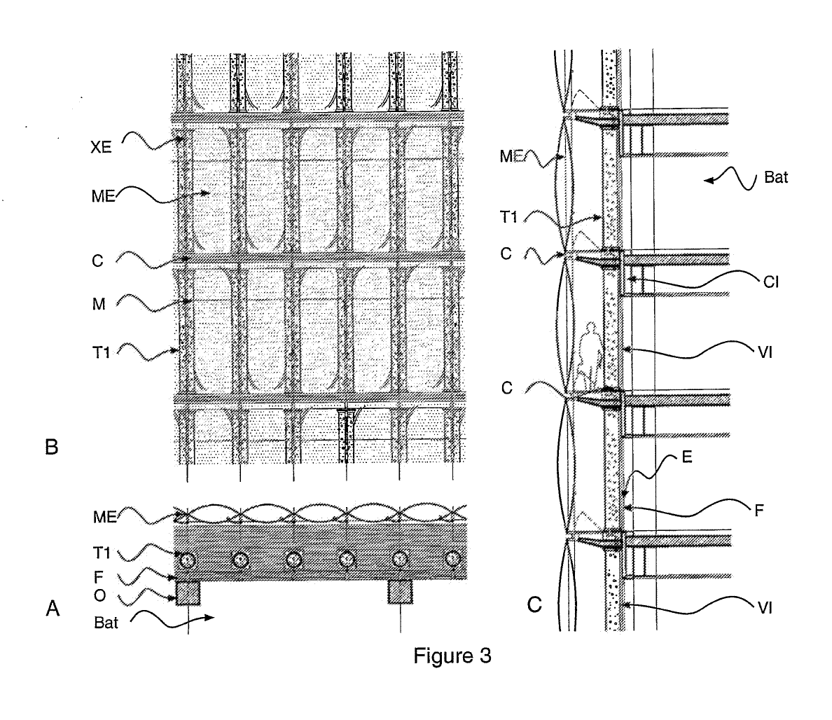

An embodiment of the invention is described here. FIG. 3 shows the device of example disposed between the frontage of a building (F) that becomes the internal envelope and an ETFE membrane, the latter forming the external envelope. The culture medium, the alga, the culture conditions, the building and the frontage are identical to those in Example 1. FIG. 3A is a plan view, 3B is a front view and 3C a transverse section.

In this example, the culture container is a vertical tube (T1). This tube is a glass tube with a diameter of 40 cm. The size of the tubes is 3.20 m as shown in FIG. 2.

The external envelope in an ETFE membrane is bolted to the slab nose of the gangway (C) by a structure made from uprights (XE) and crossmembers (YE) made from steel and bolted together. The ETFE membrane is fixed to the bearer structure by means of clamping caps that grip the membrane on four sides thereof. The clamping caps are produced from tubular profiles 5 cm wide by 3 cm deep and made from aluminium. The insulation between the outside and inside is provided by an intermediate ethylene propylene diene monomer (EPDM) seal sold by the company Alder, 9 mm wide. The gangway is identical to the one in Example 1.

The glass tube (T1) is placed on an angle bracket (A) in front of the frontage (F) of the building. The angle bracket (A) is identical to the one in example 1. The gangway (C) is sized so as to enable a person to pass and stand in front of the tube in order to maintain it and replace it where necessary. The width of the passage is a minimum of 70 cm. A person maintaining the tubes passes inside the double skin between the internal envelope and the ETFE membrane (ME).

As demonstrated in this example, the ETFE membrane also thermally insulates a building and isolates the culture content from the external air.

### Example 4

### Device According to the Invention Disposed on a Roof of a Building

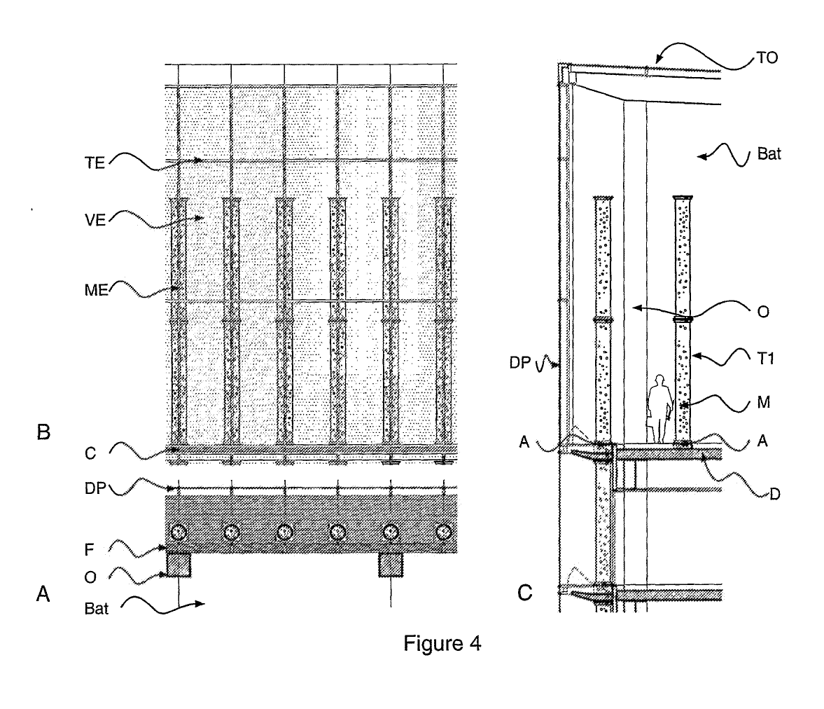

An embodiment of the invention is described here. The device used is shown schematically in FIG. 4.

The culture medium, the alga, the culture conditions, the building and the frontage are identical to those in example 1.

In this example, the tubes are fixed by screwing to the slab of the last floor of the building. The tubes are held together at the top by steel beams.

The glass roof (TO) reinforces the thermal insulation of the building and isolates the culture tube from the external air.

### Example 5

### Device with a Plate Container Fixed to a Building Frontage

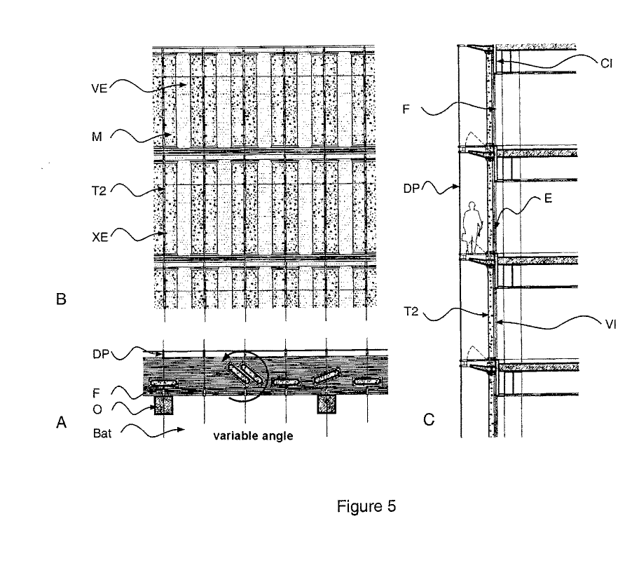

An embodiment of the invention is described here. The device used is shown schematically in FIG. 5. The culture medium, the alga, the culture conditions, the building and the frontage are identical to those in example 1.

In this example, the culture container is a vertical hollow panel (T2). This hollow panel consists of parallel glass 5 cm thick profiled with rounded corners made from profiled glass that connect the two plates together. The profiled glass part of the plate has a depth of 20 cm and a width of 90 cm. It has a height of 3.20 m.

The hollow panel (T2) is placed on a moulded rectangular piece 100 cm wide and 30 cm deep. This piece holds the glass by strapping with a moulded piece at the bottom and top, that is to say at a height of 30 cm and 320 cm respectively. The volume of medium contained in this plate is approximately 500 litres.

In this example, when the culture container is disposed against the frontage, it is fixed in the same way as example 1.

### Example 6

### Device with a Circular Culture Container Fixed to a Building Frontage

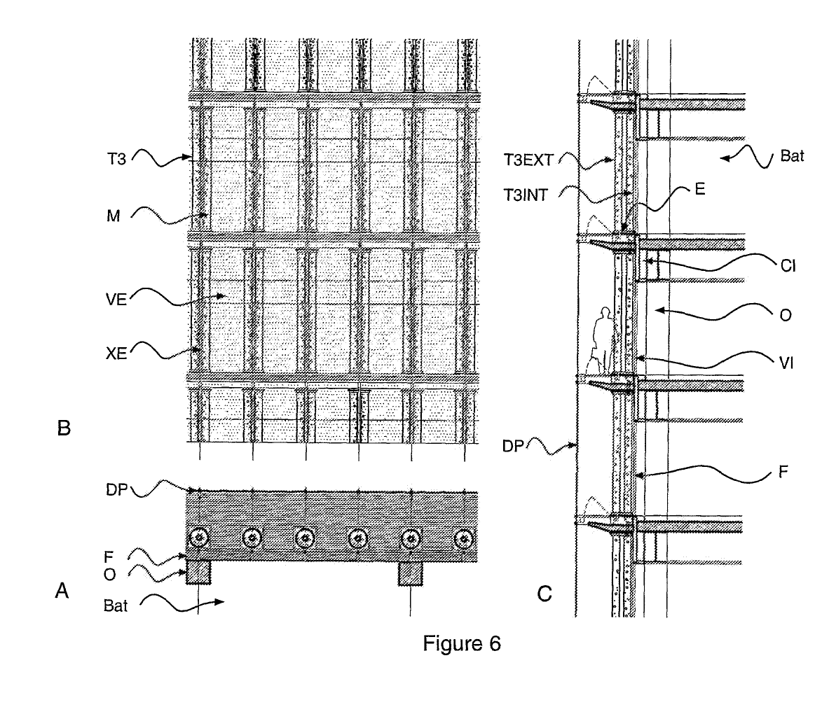

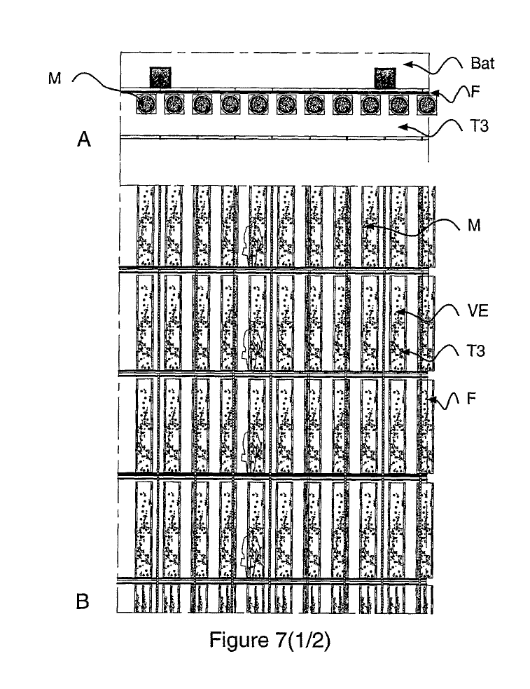

An embodiment of the invention is described here. The device used is shown schematically in FIG. 6. The culture medium, the alga, the culture conditions, the building and the frontage are identical to those in example 1.

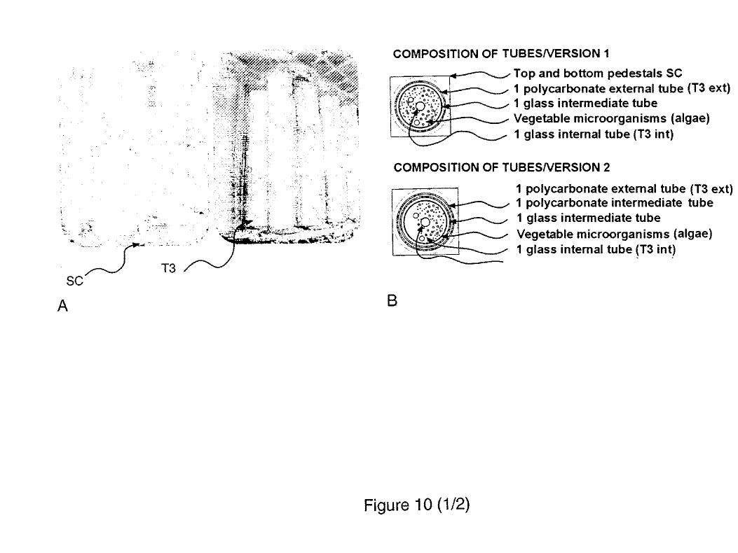

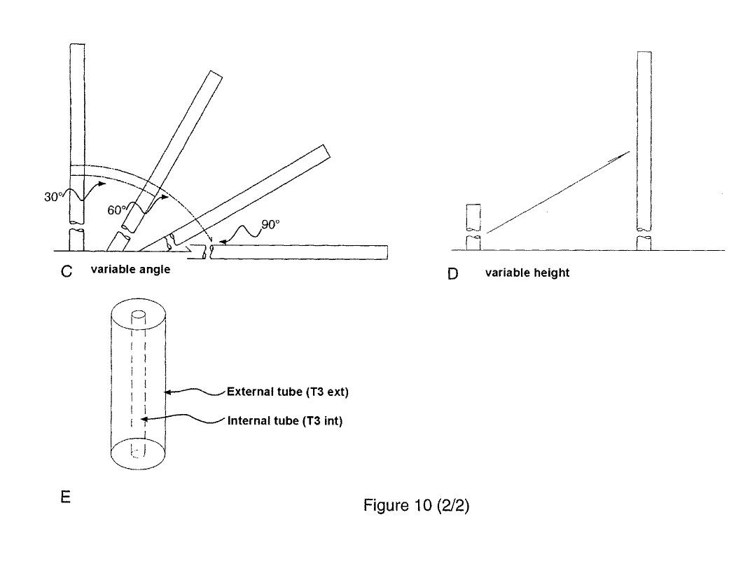

In this example, the culture container is a vertical tube (T3). This tube is a multilayer tube comprising a polycarbonate external tube (T3ext) 5 cm thick, an intermediate glass tube 5 cm thick (T3int) and an internal tube 5 cm thick (T1) made from glass, shown in FIG. 10B.

The tubes have a height of 3.20 m in FIG. 6, the external glazed tube has a diameter of 50 cm, the internal tube has a diameter of 10 cm. The tube (T3) is placed on a moulded square piece 60 cm square. This piece holds the glass by strapping with a moulded piece at the bottom and top, that is to say at a height of 3 cm and 320 cm respectively. The volume of culture medium contained in this plate is approximately 400 litres.

The internal tube (T1) comprises electrochemiluminescent diodes (C) emitting a wavelength of 630 nm.

The tubes are illuminated automatically by these diodes at night by virtue of an automatic timing system.

The culture medium lies between the external surface of the internal tube and the internal surface of the intermediate tube.

In this example, where the culture container is disposed against the frontage, it is fixed in the same way as example 1.

### Example 7

### Production and Transformation of Biomass by a Device According to the Invention Disposed on a Frontage of a Building with a Double Skin

The culture conditions and medium used in this example are identical to the one in example 6.

In this example the gaseous effluent also comprising CO₂ and NO₂ is recovered from a ventilation circuit of a building. It is routed to a set of multilayer vertical tubes with a height of 2 m and a diameter of 80 cm. The tubes comprise a polycarbonate external tube, a glass intermediate tube and a glass interior tube and a square pedestal at the top and bottom of the tube as presented in FIG. 6.

The multilayer tube comprises, between the intermediate tube and the internal tube, a culture medium identical to the one in example 1 and Chlorella algae. The gaseous effluent is injected into the medium through the bottom end of the tube via a flexible polyethylene pipe. The multilayer tube comprises, in the internal tube, luminous diodes emitting a light with a wavelength of 660 nm. The tubes are illuminated automatically by these diodes at night by means of an automatic timing system.

The tubes are disposed in front of a frontage of a building as presented in example 1.

A supply reservoir comprising culture medium is connected to the multilayer tubes via an orifice situated at the base of the container. A drainage pump is also connected to the container with a plastic tube through an orifice situated at the base of the tube. The assembly is connected independently to the tubes, thus allowing drainage and renewal of the culture medium as indicated in the aforementioned example 1.

The addition of CO₂ and NO₂ by the gaseous effluent in the culture medium is achieved as described in example 2 above.

The tubes containing the algae in the presence of sun and/or under lighting with the diodes produce organic matter and the emission of oxygen by the algae. This production of matter is done by photosynthesis by said algae. The organic matter produced is recovered by pumping 75% of the culture medium into a storage container, namely a 100 litre vessel, every four days.

The pumped culture medium is then subjected to a transformation step in which the triglycerides are extracted. These triglycerides are then subjected to a trans-esterification reaction with methanol. The product obtained is monoestermethyl corresponding to biodiesel.

In another embodiment, the algae are taken off with the culture medium and the algae are pressed cold in order to recover a larger quantity of triglycerides and therefore to produce biodiesel.

In another embodiment, the pumped culture medium is routed to a biomass thermal station, where it is consumed in order to produce energy.

In another embodiment of this example, the culture medium is subjected to pyrolysis at a temperature of between 50° and 500° C. enabling hydrocarbons to be recovered, which can be used as a biofuel. FIG. 6 shows a diagram of this example.

As demonstrated in the above examples and in this example, the arrangement of the device in front of any building frontage enables the temperature to be regulated around the culture container and insulates the building. This is because the inventors have found surprisingly that the frontage of the building has a contribution of heat to the ambient air and that it makes it possible to regulate the culture temperature of the algae. Moreover, this regulation also allows purification of gaseous effluent and the production of biomass.

This example therefore clearly demonstrates that the use of the device on a building frontage makes it possible to product organic matter by means of the algae while recycling the gaseous effluents from the building and insulating the latter. The present invention makes it possible to produce an energy bridge between the building and the culture device.

1. Device for treating an effluent and/or manufacturing a biomass, comprising:
a container for culturing alga and/or microorganisms in an aqueous medium,
a means of feeding the alga and/or microorganism culture,
a means of injecting an effluent into the alga and/or microorganism culture, said effluent coming from a building,
a means of regulating the temperature of the alga and/or microorganism culture,
optionally lighting favourable to the culture of algae and/or microorganisms, and
a means of recovering said effluent issuing from a building in order to inject it into the alga and/or microorganism culture.

2. Device according to claim 1, in which the effluent is a gaseous or liquid effluent.

3. Device according to claim 1, in which the means of recovering said effluent is chosen from the group comprising a fan, a suction pump, a ventilation circuit, an air conditioning circuit or an air filtration circuit for a building.

4. Device according to claim 1, in which the container is a container transparent to light to which the alga is sensitive for culture thereof.

5. Device according to claim 1, in which the alga and/or microorganism culture container is in a form chosen from the group comprising a cylinder, a tube, a flat tube, a tube corrugated over its length, a hollow panel, a sphere, a cube, a right-angled parallelepiped, a right-angled parallelepiped with rounded edges, a spiral, a sachet or a hollow shape without sharp edge.

6. Device according to claim 1, in which the alga and/or microorganism culture container is in the form of a tube, said tube being an alga and/or microorganism culture tube.

7. Device according to claim 6, in which the tube is a multilayer tube comprising, from outside to inside, concentrically, an external layer, a middle layer and an internal layer, and also comprising a lighting or backlighting means.

8. Device according to claim 1, in which the means of supplying the alga and/or microorganism culture comprises an automatic pump, means of regulating the supply of the algae and a supply reservoir.

9. Device according to claim 1, in which the means of regulating the temperature of the alga and/or microorganism culture is chosen from the group comprising a thermostat connected to a sensor.

10. Device according to claim 1, also comprising a means of heating the alga and/or microorganism culture chosen from the group comprising a means of recovering heat from a building, a means of recovering external heat, a means of recovering solar heat and a means of recovering heat energy.

11. Device according to claim 1, also comprising a means of cooling the alga and/or microorganism culture chosen from the group comprising a means of recovering coolness from a building, a means of recovering external coolness and means of recovering heat energy.

12. Device according to claim 10, in which the means of recovering heat from a building is a double skin of the building.

13. Device according to claim 12, in which the means of recovering coolness from a building is a double skin of the building.

14. Device according to claim 12, in which the double skin comprises an external envelope.

15. Device according to claim 14, in which the external envelope of the double skin is fixed to and parallel to a frontage of the building.

16. Device according to claim 12, in which the alga and/or microorganism culture container is positioned between said frontage of the building and said external envelope.

17. Device according to claim 12, in which the external envelope of the double skin can be impermeable or limit the passages of air coming from outside, in particular in winter, in order to protect the container from variations in temperature.

18. Device according to claim 1, in which the device also comprises a structure reinforcement for supporting the alga and/or microorganism culture container, wherein the alga and/or microorganism culture container may for example be an alga and/or microorganism culture tube, said reinforcement being able to be fixed to the building.

19. Device according to claim 1, said device also comprising at least one gangway, said gangway being able to serve as a support for the alga and/or microorganism culture container and/or as a means of access to the alga and/or microorganism culture container for maintenance personnel.

20. Device according to claim 2, in which said effluent is a gaseous effluent chosen from the group comprising CO₂, nitrogen dioxide or air vitiated by human occupation, by the circulation of vehicles in the building (car parks) or in the vicinity of the building (tunnels, underground roads, spaces under slabs), or by industrial production.

21. Device according to claim 2, in which the liquid effluent is a liquid effluent chosen from the group comprising a liquid containing a metal and a liquid containing a polluting substance.

22. Device according to claim 1, in which the biomass is an oily biomass.

23. Device according to claim 1, in which the biomass is a biomass that can be converted into coal or biopetroleum.

24. Device according to claim 1, in which the device is integrated in the structure of a building or attached to the building.

25. Device according to claim 1, in which the building is an industrial and/or office and/or dwelling and/or agricultural building and/or a civil engineering and/or mixed structure.

26. Device according to claim 1, in which the algae are chosen form the group comprising Chlorophyceae, Chlorella, Parietochloris incisa, Amphora sp., Nitzchia sp. and Chaetoceros sp. diatoms, and Chrysophyceae.

27. Device according to claim 1, said device also comprising a means of recovering biomass formed by the culture of algae and/or microorganisms.

28. Device according to claim 1, said device also comprising a system of draining the alga culture container.

29. Device according to claim 1, said device also comprising one or more of the following means of controlling and regulating the culture of alga: means of controlling the supply of algae and/or microorganisms, means of controlling the injection of effluent to be treated, means of controlling the temperature, means of controlling the pH and means of controlling the lighting of the algae and/or microorganisms.

30. Device according to claim 1, said device being controlled by computer in order to optimise the culture of the algae and/or microorganisms and/or the treatment of the effluent.
